(19) United States
(12) Patent Application Publication
(10) Pub. No.: US 2025/0195636 A1
(43) Pub. Date: Jun. 19, 2025

(54) RSV PASSIVE AND ACTIVE VACCINES

(71) Applicant: Trellis Bioscience, Inc., Redwood City, CA (US)

(72) Inventors: Milton J. Friedman, Alameda, CA (US); Lawrence M. Kauvar, San Francisco, CA (US)

(73) Assignee: Trellis Bioscience, Inc., Redwood City, CA (US)

(21) Appl. No.: 19/028,538

(22) Filed: Jan. 17, 2025

Related U.S. Application Data
(63) Continuation of application No. 17/168,553, filed on Feb. 5, 2021, now Pat. No. 12,246,063.
Application No. 17/168,553 is a continuation of application No. PCT/US2019/045775, filed on Aug. 8, 2019.
(60) Provisional application No. 62/716,184, filed on Aug. 8, 2018.

(51) Int. Cl. A61K 39/12; A61K 39/00; A61P 31/14

(52) U.S. Cl. CPC A61K 39/12; A61P 31/14; A61K 2039/545; A61K 2039/6056

(57) ABSTRACT

Improvements to vaccines against RSV include G protein CCD portions complexed with mAb that block interaction with CX3C-R and modified forms of mAb to prolong serum half-life, as well as inhalable vaccines.

### CROSS-REFERENCE TO RELATED APPLICATIONS

This application is a continuation of and claims priority to co-pending U.S. patent application Ser. No. 17/168,553 filed Feb. 5, 2021, and claims the benefit under 35 U.S.C. § 120 to, International Application No. PCT/US2019/045775, filed Aug. 8, 2019, and now expired which claims priority to now expired U.S. provisional application 62/716,184 filed 8 Aug. 2018, the contents of which are incorporated herein by reference in their entirety.

### INCORPORATION BY REFERENCE OF SEQUENCE LISTING

The application herein incorporates by reference in its entirety the sequence listing material submitted concurrently with the specification as an XML file, with a filename of “12774-138US2” a creation date of Jan. 13, 2025 and a size of 45056 bytes. The ST26 Sequence Listing is part of the specification and is incorporated in its entirety by reference herein.

### FIELD OF THE INVENTION

The invention is directed to improved methods and materials for prophylaxis and treatment of respiratory syncytial virus (RSV) infection.

### BACKGROUND ART

RSV is a negative-strand RNA virus with 10 genes encoding 11 proteins, which has resisted effective management for over 60 years in part because infection does not provide robust immunity (Broadbent, L. et al. Influenza Other Respir Viruses (2015) 9:169-78). To date, no vaccine has been approved despite several substantial attempts (Jorquera, P.A. and Tripp, R.A. Expert Rev Respir Med (2017) 11(8):609-615). Over 50% of infants in the US are infected during their first year, with nearly 5% requiring hospitalization (American Academy of Pediatrics Committee on Infectious Diseases, American Academy of Pediatrics Bronchiolitis Guidelines. Pediatrics (2014) 134:e620-38). Severe RSV disease in infancy is an established risk factor for childhood asthma-like symptoms (Gelfand, E.W. Curr Opin Immunol (2012) 24(6):713-9). Preterm infants (<29 weeks gestational age) who are at particularly high risk have been the focus for prophylaxis with a humanized mouse monoclonal antibody or mAb (palivizumab, marketed by Medlmmune as Synagis®), an expensive treatment which reduces morbidity but not mortality (Meissner, H.C. and Kimberlin, D.W. Pediatrics (2013) 132:915-8). Global incidence of RSV infection in young children is less well documented than in the US but is believed to be more than double that of US, with higher mortality rates (Nair H., et al. Lancet (2010) 375(9725):1545-1555). In addition, up to 12% of medically attended acute respiratory illness in older adults is attributed to RSV infection with 6-8% of these cases being lethal. Hospitalizations last 3 to 6 days, with a substantial fraction admitted to the intensive care unit (Colosia, A.D., et al. PLoS ONE. (2017) 12(8):e0182321). RSV infections are also common in immunocompromised patients, with mortality of over 25% (Shah, D.P., et al. Blood (2014) 123(21):3263-3268).

Two subtypes of RSV, A and B, circulate as the predominant strains alternately at ˜1-2-year intervals, with comparable incidence worldwide. The two subtypes comprise different genotypes, and can co-circulate during an infection. Thus, an effective vaccine or treatment needs to show activity on strains from both subtypes.

RSV has two major surface glycoproteins, F and G. Currently, the sole marketed mAb against RSV is palivizumab (commercially available under the brand name Synagis®) which is only approved for prophylactic use in premature infants, and is directed against the F protein. This mAb is broadly useful due to conservation of the F protein sequence among strains, although escape mutations are readily detected (Zhu, Q., et al. J Infect Dis (2011) 203:674-82). Efforts to develop a vaccine based on the RSV F protein have been described (Jorquera, P.A. and Tripp, R.A. Expert Rev Respir Med (2017) 11(8):609-615). Although antibody titers to the F protein produced in immunized subjects have reached levels that inhibit RSV propagation in cell culture, RSV disease has not been sufficiently prevented for any such vaccine to receive FDA approval.

Further, palivizumab treatment of premature infants (born at 32-35 weeks gestation) did not have a major effect on asthma or lung function at age 6 years (Scheltema, N.M., et al. Lancet Respir. Med. (2018) 6:257-264). Escape mutations from palivizumab are well documented and may contribute to the lack of efficacy (Bates, J.T., et al. Virology (2014) 454-455:139-44). Moreover, a higher affinity derivative of palivizumab, motavizumab, failed to show a clinical benefit as a treatment of acute infection in full term infants, even when given at the very high dose (for a mAb) of 100 mg/kg (Ramilo O., et al. Pediatr Infect Dis J. (2014) 33(7):703-9).

By contrast, the G protein overall is quite variable among strains. However, a region in the middle of the sequence including residues 167-176 is very highly conserved, and is denoted as the central conserved domain (CCD). In fact, <1% of ˜7,000 published RSV sequences show any changes in the CCD, with the majority being conservative substitutions.

Initial attempts at prophylaxis for RSV by vaccination with formalin-inactivated virus proved counterproductive, leading to enhanced disease and pulmonary eosinophilia (Kim, H.W., et al. Am J Epidemiol (1969) 89:422-434). This harmful reaction now appears to result from an interaction of RSV G protein with the CX3CR1 receptor, resulting in recruitment and possible activation of inflammatory cells. The region that interacts with this receptor, the CX3C motif is at positions 182-186, just outside the CCD. As a result of this observation the formalin inactivated vaccine has not been further developed. Moreover, RSV G protein has been shown to exacerbate RSV infection by depressing the interferon response (Oshansky, C.M. et al. Viral Immunol (2009) 22(3):147-161). Vaccines based on variants of RSV G protein but lacking the CX3CR1 interaction have been proposed. U.S. Pat. No. 8,173,131 discloses vaccines wherein the CX3C motif is modified or compositions that comprise antibodies or other moieties that prevent interaction of the motif with the receptor, thus preventing cell entry. U.S. Pat. No. 8,846,056 discloses immunogenic peptides from RSV G protein as vaccine components, specifically including residues 164-176 or 155-206. U.S. Pat. No. 9,321,830 discloses mAbs useful in treating or preventing RSV infections that bind to conserved linear sequences of the G protein of RSV in the region including residues 167-176; as these antibodies were derived from the natural human immune repertoire, they are expected to be minimally immunogenic when administered to a human subject.

About 15% of the RSV G protein is secreted due to use of an alternative translation start site at codon 48 that eliminates the cytoplasmic domain and a portion of the transmembrane domain region (Hendricks, D.A., et al. J Virol (1988) 62:2228-33). Deletion of this start site considerably reduces virulence (Stobart, C.C., et al. Nat Commun (2016) 7:13916), (Arnold, R., et al Virology (2004) 330:384-397). Such an attenuated live virus may be useful as a vaccine, although the high transmissibility of RSV poses a substantial risk of transfer to people at risk for severe disease despite the attenuation, including the very young, the elderly, and the immunocompromised. The secreted G protein poses an additional obstacle to an effective vaccine or treatment, because high affinity for antigen is needed for neutralizing soluble factors; otherwise the mAb simply provides a circulating reservoir for the antigen, extending its serum half-life and distributing it more efficiently around the body (Tabrizi, M., et al. AAPS J (2010) 12:33-43). As a result, mAbs with low picomolar (pM) affinity for the RSV G protein CCD, such as those disclosed in U.S. Pat. No. 9,321,830 are generally advantageous.

Collarini, E.J., et al. J Immunol (2009) 183:6338-45 describes high affinity, broadly neutralizing mAbs that bind to the RSV G protein CCD as well as antibodies with varying affinities including 3D3 (1.1 pM), 2B11 (10 pM), 3G12 (580 pM), 5D8 (4.4 nM). The mAb 3D3 has shown broad neutralizing activity both in vitro and in rodent models; the epitope for 3D3 defined at the level of short linear peptides is highly conserved across nearly all circulating strains. An additional mAb, 2D10, is of interest, because no linear peptide epitope could be defined for it although it is active in viral neutralization assays in vitro, albeit with lower potency than 3D3 (U.S. Pat. No. 8,273,354). Additional native human mAbs with affinities in the low nM to high pM range are disclosed in U.S. Pat. No. 10,035,842.

As disclosed in US Publication No. US-2019-0135876-A1, the structures of peptides from the CCD of the G protein complexed with mAbs 3D3 and 2D10 have been determined at high resolution by x-ray crystallography and the conformational character of the epitopes thereby defined.

A recombinant fusion protein comprising the central region (residues 131-230) of the G proteins of both RSV A and B subtypes was shown to be an effective immunogen in mice (Lee, J.Y. and Chang, J. PLoS One (2017) 12:e0175384). A G protein peptide, residues 148-198 emulsified in DMSO and PBS, has also been tested in mice as an immunogen and found to be effective at inducing antibodies that both neutralize infection and block G protein binding to CX3CR1 (Choi, Y., et al. Viral Immunol (2012) 25(3):193-203). Similarly, vaccination with nanonparticles that incorporate the G protein residues 169-198 induced protection in mice challenged with RSV (Jorquera, P. A., et al. PLoS One (2013) 8:e74905). Fine scale optimization of peptides from this region has also been attempted, with insertion of one extra residue into the G protein at residues 182-186 greatly reducing the deleterious effects of G protein on the host immune response (Boyoglu-Barnum, S., et al. J Virol (2017) 91(10) pii: e02059-16) but likely also reducing the ability to induce mAbs against the native CCD.

The contents of these documents and all documents cited herein are incorporated herein by reference.

Although the results to date are encouraging, no RSV G protein vaccine has been approved. Repeated infection is common for this virus, unlike many others, with increased severity in many instances. Criteria defining quality of an immunogen, for which further improvements are useful, include: duration of response; uniformity of response (titer and affinity) across a diverse human population; broad spectrum (active against all circulating strains); safety (in particular, lack of deleterious pharmacological activity on the CX3C chemokine receptor); and stability (particularly important for use in countries lacking an effective refrigerated supply chain). Prevention of viral disease by vaccination usually involves subdermal or intramuscular injection of either intact virus particles, disabled virus particles, or some portion of a virus particle combined with additional components called adjuvants. Adjuvants are added to vaccines for the purposes of enhancing vaccine efficacy, stability, and preservation. Antibodies may be administered intravenously, intramuscularly, or by inhalation.

Use of immune complexes as vaccines can enhance the primary immune response (both kinetically and quantitatively) compared to vaccination with antigen alone (Houston WE, et al. Journal of Infectious Diseases (1977) 135(4):600-10), (Brady, L.J. Infection and Immunity (2005) 73(2):671-8). This is attributed to increased efficiency in antigen processing and presentation (Abdel-Motal. U.M., Wigglesworth. K, Galili. U. Vaccine (2009) 27(23):3072-82). Inclusion of anti-antigen antibody complex in vaccines has been found in other systems to boost vaccine effectiveness by stimulating formation of immune complexes which in turn stimulate antigen-specific immune responses (Lambour, J. et al. Emerging Microbes & Infections (2016) 5:e92; doi: 10.1038/emi.2016.97).

Improvements in key features of both active and passive vaccines targeting the G protein is the focus of the present invention. Although efforts with regard to RSV immunization have focused on the F protein, it is now understood that the G protein is an important target and it can be effectively adopted as an immunogen for improved RSV prophylaxis.

### DISCLOSURE OF THE INVENTION

In one aspect, the present invention enables the conserved CCD of the RSV G protein to elicit neutralizing antibodies in a subject while eliminating the undesired interaction of the CX3C motif with its receptor. This is accomplished by mutating the G protein to prevent this interaction or by providing antibodies or other moieties that compete with the G protein for this receptor. According to the present invention, an anti-G mAb is used to bind RSV G protein in a vaccine composition. The antibodies employed in the complexes of the present invention prevent the interaction of the RSV G protein with the host cell receptor CX3CR1, thereby preventing adverse events tied to that biological activity, without interfering with the ability of the G protein or the CCD portion thereof to induce neutralizing antibodies. Such a combination has been shown to block the activation of the CX3CR1-receptor (see Example 4 below), and such complexes are able to increase efficiency of immune response as explained below.

Thus, in one aspect, the invention relates to an immunogen and vaccine compositions that contain it that comprise RSV G protein or a fragment comprising the CCD, or a homolog or stabilized forms thereof or peptidomimetic of either, complexed with a mAb (or fragments or derivative thereof) that binds to RSV G protein with high affinity and prevents interaction with CX3CR1. The immunogen may be administered along with a vaccine adjuvant. The CCD is that of residues 131-230 or 167-176 of a G protein. To be effective in this aspect, the mAb needs to have high enough affinity to support formation of stable immune complexes, such as an affinity equal to or better than that associated with a Kd of 100 pM or 50 pM or 25 pM or less.

In another aspect, the invention includes methods to provide prophylaxis with respect to RSV infection in a subject which method comprises administering to a subject in need of such treatment a pharmaceutical or veterinary composition including these immunogens.

In another aspect, the invention includes sequence modifications to a mAb against the CCD of the RSV G protein wherein the modifications result in extended serum half-life. Technology for achieving this goal has long been known, as described in U.S. Pat. No. 8,318,907. Targeting the G protein in this fashion is a contribution to passive immunotherapy/prophylaxis against RSV, wherein the selection of antigen to target is important. That is, the vast majority of vaccine/antibody efforts on RSV have been focused on the F protein, with no prior suggestion that the long half-life approach currently being applied to the F protein should or could be applied to the G protein. Other techniques for extending the half-life include fusion to an albumin binding domain or modification of the Fc region to enhance binding to mucin.

In yet another aspect, the invention includes expression in the cells of a subject of the mAb by Vectored Immunoprophylaxis (VIP), also referred to as Immunoprophylaxis by Gene Trasfer (IGT) or Vector-Mediated Antibody Gene transfer, which comprises introducing genes encoding a mAb into the subject's cells which then secrete the mAb (Sanders, J.W. and Ponzio, T.A. Tropical Diseases, Travel Medicine and Vaccines (2017) 3:3).

In another aspect, the invention includes formulation of a mAb against the CCD of RSV G protein that enables delivery by inhalation. Suitable technology to achieve this goal is described in U.S. Pat. No. 9,718,875. A review of suitable nebulizers is: (Ari, A. and Fink, J.B. Curr Pharm Biotechnol. (2016) 17(14):1268-1277).

### MODES OF CARRYING OUT THE INVENTION

The conformational epitopes on RSV G CCD defined by high affinity, broadly neutralizing mAbs 3D3 and 2B10 disclosed in U.S. Pat. No. 8,273,354, incorporated herein by reference include helices, disulfide bonds, and polar and hydrophobic interactions between discontinuous amino acids. Similar results have been described using additional mAbs against the G CCD (Jones H.G., et al. PLoS Pathog (2018) 14(3):e1006935). These features may explain why linear RSV G epitope peptides have not been fully effective as antigens; for example, an early attempt to target the CCD of the G protein with a recombinant protein vaccine (BBG2Na) showed only a moderate ability to induce neutralizing antibodies in healthy, young adults and a more recent effort also using recombinant G protein failed to establish efficacy in elderly adults (Rezaee, F., et al. Curr Opin Virol (2017) 24:70-78).

As described in Publication No. US-2019-0135876-A1 these conformational features can be stabilized by use of chemical crosslinkers or non-natural amino acids (Robinson, J.A. J Pept Sci (2013) 19(3):127-40). The contact residues with the efficacious mAbs comprise most of the CCD but not all. Mutations in the residues not involved in mAb binding may thus be mutated to prevent interaction of the immunogen with CX3CR1 without affecting the mAb binding, or as employed in the present invention the site responsible for binding CX3CR1 can be masked by binding to an mAb or fragment thereof (such as those noted above) so as to prevent interaction with this receptor. This is an advantage over and above the enhanced immunogenicity of the complex described for such complexed immunogens.

### Definitions

Unless defined otherwise, all technical and scientific terms used herein have the same meanings as commonly understood by one of ordinary skill in the art to which this disclosure belongs. Detailed methods for constructing many of the reagents is provided in Example 1 herein below.

“Protein”, “peptide” and “polypeptide” are used interchangeably and to refer to chains of naturally occurring amino acids coupled through amide bonds such that they can be synthesized by recombinant methods regardless of the length of the chain. “Homologs” are peptides or proteins with similar sequences, but alterations such that the homolog is 80% or 85% or 95% or 99% homologous to the referent. “Peptidomimetics” have similar homologies but include unnatural or synthetic amino acids, including D and L isomers and amino acid analogs linked by amide linkages or other bonds, e.g., ester, ether, etc. “Peptidomimetics” also include organic molecules not obviously analogous to peptides, including, for example, aptamers. As defined herein, based on previous studies, the CCD of the G protein is that portion of the protein represented by residues 169-198.

As used herein, “subject” refers to a human or non-human animal, including laboratory models for RSV such as rodents, or to livestock or pets.

As used herein, “binding moiety” includes antibodies and alternative non-immunoglobulin binding moieties as set forth hereinbelow. “Antibodies” include immunoreactive fragments of traditional antibodies and their various fragmented forms that still retain immunospecificity such as Fab, F(ab′)₂, Fv fragments, single-chain antibodies in which the variable regions of heavy and light chain are directly bound without some or all of the constant regions. Since light chains are often interchangeable without destroying specificity, antibodies composed of a heavy chain variable region that determines the specificity of the antibody may be combined with a heterologous light chain variable region. Chimeric antibodies with constant and variable regions derived, for example, from different species are also included. Also included are antibodies in which the Fc portion of the molecule has been mutated to enhance or reduce binding to Fc receptors (Kontermann, R.E. BioDrugs (2009) 23(2):93-109).

For the variable regions of mAbs, as is well known, the critical amino acid sequences are the complementarity-determining region (CDR) sequences arranged on a framework, which framework can vary without necessarily affecting specificity or decreasing affinity to an unacceptable level. Definition of these CDR regions is accomplished by art-known methods. Specifically, the most commonly used method for identifying the relevant CDR regions is that of Kabat as disclosed in Wu, T. T., et al., J. Exp. Med. (1970) 132:211-250 and in the book Kabat, E. A., et al. (1983) Sequence of Proteins of Immunological Interest, Bethesda National Institute of Health, 323 pages. Another similar and commonly employed method is that of Chothia (Chothia, C., et al., J. Mol. Biol. (1987) 196:901-917) and (Chothia, C., et al., Nature (1989) 342:877-883), including additional modifications (Abhinandan, K. R., et al., Mol. Immunol. (2008) 45:3832-3839). The mAbs described herein include the CDR regions as defined by any of these systems or other recognized systems known in the art.

The specificities of the binding of mAbs are defined, as noted, by the CDR regions mostly those of the heavy chain, but complemented by those of the light chain as well (the light chains being somewhat interchangeable). Therefore, the mAbs of the invention may contain the three CDR regions of a heavy chain and optionally the three CDR's of a light chain that matches it. Because binding affinity is also determined by the manner in which the CDR's are arranged on a framework, the mAbs may contain complete variable regions of the heavy chain containing the three relevant CDR's as well as, optionally, the complete light chain variable region comprising the three CDR's associated with the light chain complementing the heavy chain in question. Preferred CDR's for the mAbs of the present invention are disclosed in FIGS. 5A and 5B of U.S. Pat. No. 8,273,354. In particular, mAbs of the invention may include variable regions of the heavy and light chains of mAb 3G12, 3D3, 2B11, or 2D10, i.e., SEQ ID NO: 2 and 16; SEQ ID NO: 4 and 18; SEQ ID NO: 6 and 20; SEQ ID NO: 8 and 22, respectively.

The invention also includes binding moieties that mimic the binding characteristics of mAbs. Suitable mAb mimics include aptamers (Yu, Y., et al. Int J Mol Sci (2016) 17(3):358) and protein mimics of antibodies or fragments thereof (alternative scaffolds) such as camelids, anticalins, ankyrin repeat proteins (Azhar A., et al. Int J Biol Macromol (2017) 102:630-641).

An alternative to active immunization is to provide a mAb as passive immunotherapy, either prophylactically or therapeutically. For prophylaxis, it is useful to modify the Fc region of the mAb to extend serum half-life as has been described for an anti-F protein mAb now in clinical development (U.S. Pat. No. 7,323,172). For therapeutic use, an inhaled formulation is useful, as has been described for mAbs against influenza (U.S. Pat. No. 9,718,875).

### Recombinant Aspects

Any proteins or peptides of the invention including antibodies or antigen binding fragments thereof may be produced recombinantly using known techniques. The invention also includes nucleic acid molecules comprising nucleotide sequences encoding them, as well as vectors or expression systems that comprise these nucleotide sequences, cells containing expression systems or vectors for expression of these nucleotide sequences and methods to produce the peptides by culturing these cells and recovering the binding moieties produced. Any type of cell typically used in recombinant methods can be employed including prokaryotes, yeast, mammalian cells, insect cells and plant cells. Also included are human cells (e.g., muscle cells or lymphocytes) transformed with one or more recombinant molecules that encode the relevant peptides.

### Activities Based on the CX3C Chemokine Motif

Although variable overall, RSV G (298 residues) contains an approximately 40 amino acid central conserved domain (CCD) that is highly conserved, and devoid of glycosylation; this portion of the protein has been shown to play key roles in both virus infection and viral pathogenesis. Specifically, RSV G CCD contains a CX3C chemokine motif that facilitates binding to the human chemokine receptor CX3CR1 to promote RSV infection in human airway epithelial cells as well as modulating signaling that affects trafficking of CX3CR1⁺ immune cells resulting in airway congestion (Tripp, R.A., et al. J Virol (2018) 92:e01302-17).

Aside from the presence of a CX3C motif and its two disulfide bonds there are no structural or sequence similarities between RSV G and fractalkine/CX3CL1, the only known ligand for CX3CR1. This structural divergence despite similar functionality provides an opportunity to develop therapies that selectively block the viral-host interaction, a strategy that led to an antagonist of the HIV co-receptor CCR5 (Lieberman-Blum, S.S., et al. Clinical Therapeutics (2008) 30:1228-1250).

The affinity of the stabilized forms of RSV G protein CCD or peptidomimetics thereof and their spatially constrained forms can be tested using the ELISA assays described in Example 2 or using one of the many alternative methods known in the art. Assays for induction of efficacious antibodies can be tested, using the assays set forth in Example 3 herein below, or other suitable tests well known to one of ordinary skill in the art of vaccines. The ability to activate or inhibit the CX3C receptor can be assessed using, for example, the chemotaxis assay set forth in Example 4 herein below or any other suitable method, such as a calcium flux assay that has shown good correlation with chemotaxis in a study of mutants of the endogenous ligand for CX3CR1 (fractalkine) (Dorgham, K. et al. J Leukoc Biol (2009) 86(4):903-11).

### Immunogens

Three design goals dominate optimization of a G protein immunogen.

First, the pharmacological activity of the G protein is deleterious in the context of RSV infection and it is thus preferable to minimize that activity in the immunogen either by modification of the immunogen structure as previously described or by masking that site as disclosed herein.

Second, high affinity antibodies are needed to neutralize the deleterious soluble G protein produced by virus infected cells, and therefore antibodies generated by the immunogens preferably have these properties. The method of the present invention maximizes the likelihood of achieving this goal by minimizing alterations to the native structure. Optimization of the ratio of mAb to antigen in an immunogen complex is also useful, because an excess of mAb discourages formation of immune complexes and thus reduces the efficacy of the mAb-antigen complex as an immunogen (Manca, F., et al. J Experimental Medicine (1991) 173(1):37-48).

Third, since RSV is an important pathogen worldwide, including in countries that lack a refrigerated supply chain for delivery of vaccines, stabilization of the immunogen to allow transport and storage at room temperature (or above) is also desirable. Formalin inactivation of live virus, which is effective in other vaccines, is not acceptable for RSV since the first such vaccine caused disease exacerbation upon subsequent natural infection (Kim, H.W., et al. Am J Epidemiol (1969) 89:422-34).

With respect to this third aspect, conformational stability achieved by stabilization of the structure using mutations chosen based on antibody-antigen high resolution structural data, can result in higher titer more uniformly across immunized subjects than for the parental virus, as shown for the RSV F protein (Mcclellan, J.S., et al. Science (2013) 340(6136):1113-1117). The natural G protein CCD is poorly immunogenic, and thus modifications to improve immunogenicity are important. Since the G protein CCD can be made either synthetically or recombinantly, methods well known in the art can be used to systematically mutate this peptide. To facilitate evaluation of a large number of such variants, in vitro assays are needed. Assays for thermal stability known in the literature include observation of increased fluorescence of a dye when bound to hydrophobic sites exposed as the protein unfolds (Biggar, K.K., et al. BioTechniques (2012) 53:231-238) and observation of secondary structure character by circular dichroism (Kelly, S.M. and Price, N.C. Biochim Biophys Acta (1997) 1338(2):161-185).

In addition to the disadvantage of requiring refrigeration, the use of flexible peptides as immunogens often elicits antibodies that bind weakly (≥micromolar Kd) to conformational epitopes in folded proteins. For that reason, conformationally constrained synthetic epitope mimetics are of particular interest in immunogen design, with examples including efforts addressing HIV, hepatitis C, influenza, and others (Robinson, J.A. J Pept Sci (2013) 19(3):127-40).

Peptides incorporating non-natural motifs are often quite resistant to proteolytic degradation, which is an advantageous feature unrelated to the mimicry itself. A disadvantage of small molecules (“haptens”) is that they are often not immunogenic themselves; however, they can become effective immunogens when presented to the immune system embedded in virus like particles (Buonaguro, L., et al. Exp. Rev. Vaccines (2011) 10:1569-1583).

In particular, the F protein of RSV has been subjected to such mimicry. In this instance, two “staples” (crosslinks) were required to create an effective mimic, which displayed nanomolar potency for competitive inhibition of RSV infection in Hep-2 cells in vitro (Gaillard, V., et al. Antimicrob Agents Chemother. (2017) 61(4) pii: e02241-16).

### Extended mAb Dosing

Still another aspect of the invention is providing a passive vaccine wherein an antibody directed to the RSV G protein is modified to provide an extended time period of effective lifetime in a subject or wherein the lifetime is extended by conjugation to albumin or wherein the antibody or antigen binding portion is expressed in the cells of the subject.

RSV is a seasonal virus, with a typical season lasting from October to April in the US. Over that time period, among children younger than 5 years old, there are 2.1 million outpatient visits and 57,527 hospitalizations (Hall, C.B., et al. New Engl J Med. (2009) 360(6):588-98). For adults older than 65 years, there are 177,000 hospitalizations and 14,000 deaths (Falsey, A.R., et al. New Engl J Med. (2005) 352(17):1749-59). The advantage of an extended half-life antibody for RSV to provide protection throughout the season is thus well recognized in the field.

In one embodiment, the Fc region of the mAb is modified to enhance the binding of the Fc region of IgG1 to the neonatal Fc receptor (FcRn). An Fc modified mAb against the RSV F protein (MEDI8897) is in clinical development by Sanofi in partnership with AstraZeneca (Medimmune) (ClinicalTrials.gov Identifier: NCT02290340). This mAb has 100-fold higher potency in vitro as compared to the only approved mAb targeting RSV (palivizumab). The mAb has been engineered with a triple-amino-acid (M252Y/S254T/T256E [YTE]) substitution within its Fc region (Griffin, M.P., et al. Antimicrob Agents Chemother. (2017) 61(3):e01714-16). The YTE substitution enhances the binding of IgG1 to the FcRn under the acidic conditions (pH 6.0) of the lysosome. This prevents degradation and increases recirculation to the surface of the cell, thereby prolonging the serum half-life of the antibody. The combination of higher potency and longer half-life enables a single dose to provide neutralizing activity for 3-4 months. While precedent supports the expectation of utility for an extended half-life mAb, the natural history of RSV in infants includes a rebound in viral load (of 2-3 logs) about 2 weeks post-infection, which occurs in about one third of infants (Brint, M.E. et al. Pediatr Res (2017) 82(5):872-880). This result is thought to reflect escape from the initial natural antibody response to the virus. Accordingly, for an extended half-life mAb to be maximally effective, it needs to target a functionally critical site on the virus structure. Antibodies targeting the RSV G protein, and in particular the CCD of the G protein, meet this criterion far better than antibodies targeting the F protein. Thus, the YTE substitution is applied to the mAb of the invention, such as 3G12, 3D3, 2B11 and 2D10 to provide more effective neutralization of the virus.

In another embodiment, the Fc region of the mAb is modified to bind to mucus components. Thus is of particular utility for inhaled formulations (Wessler, T., et al. ACS Infect. Dis. (2016) 2(1):82-92). Another option for extending the half-life of a mAb or fragment thereof is to fuse or conjugate the sequence to an albumin binding domain (Malm, M., et al. Biotechnol J. (2014) 9(9):1215-22).

In still another option the mAb or antigen binding portion is expressed in the cells of the subject by Vectored Immunoprophylaxis, a process in which genes encoding previously characterized neutralizing antibodies are vectored into the subject's cells which then secrete the monoclonal antibodies encoded by those genes. The technology has been proven effective in animals and is under consideration for providing extended protection in people against HIV (Sanders, J.W. and Ponzio, T.A. Tropical Diseases, Travel Medicine and Vaccines (2017) 3:3).

In one implementation, a vector comprising self-complementary adeno-associated virus (scAAV) of serotype 8 (low incidence of antibody response) supported long-lived expression of full-length human antibodies driven from CMV promoters after administration through a single injection of the gastrocnemius muscle. Within one week, antibody gene expression was detectable, achieving maximum levels after 12-16 weeks and then decreasing two-to three-fold before stabilizing for the duration of the 64-week study at >50 μg/mL of mAb. When this system was used to express a broadly neutralizing HIV antibody, mice were protected from repeated HIV infection by both IV and vaginal exposure routes for at least 15 weeks (Balazs, A.B., et al. Nature (2012) 481(7379):81-84; Balazs, A.B. et al. Nature Medicine (2014) 20(3):296-300).

In another implementation, a plasmid encoding the genes for the heavy and light chain of an antibody was introduced by electroporation into mouse muscle pre-treated with hyaluronidase to improve plasmid access to the cells (Yamazaki, T., et al. Vaccines (2018) 6(3) 35). Plasmid vectors are considered to be safer than AAV, are easy to prepare and stable during storage. Moreover, plasmid DNA does not induce an immune response against itself. An antibody against the HA protein of influenza induced by this means in mice produced >10 μg/mL of the antibodies in serum for at least 70 days following antibody gene transfer, significantly higher than the level of HA-specific IgG antibody induced from vaccination (1-3 μg/mL).

### Inhaled Formulation

The only approved antiviral drug for RSV, ribavirin, is rarely used due to its toxicity and weak efficacy (Simoes, E.A.F., et al. Infect Dis Ther. (2018) 7(1):87-120). Accordingly, palliative care, notably including mechanical ventilation, is a common technique for managing severe RSV disease in both infants and the elderly. Incorporating an antiviral antibody into the treatment by inhaled delivery is thus compatible with standard care. Commercially available mesh nebulizers are suitable for generating droplets containing antibody in the size range (low micrometer diameter) appropriate for distribution throughout the lung (Respaud, R., et al. J Control Release (2016) 234:21-32). The reduced dose for comparable efficacy as compared to systemically delivered antibody is particularly advantageous for treating the elderly due to the >10-fold higher systemic dose required for adults as compared to infants.

The RSV G protein CCD binding to CX3CR1 has been established as the dominant route of infection in human airway epithelial cells (Jeong, K.I., et al. PLoS One (2015) 10:e0130517; Johnson, S.M., et al. PLoS Pathog (2015) 11:e100531). Accordingly, delivery of the mAb to the airways will provide direct neutralization of virus shed into the airways from the apical surface of the infected cells (Villenave, R., et al. Proc. Natl Acad Sci USA (2012) 109:5040-5045; Wright, P.F., et al., J Virol (2005) 79:8651-8654; Zhang, L. et al., J Virol (2002) 76:5654-5666). Systemically delivered antibody, by contrast, attacks the basolateral side of the cells and is thus less efficient for neutralizing the virus. An inhaled antibody mimic (“nanobody” derived from a camelid antibody) targeting the RSV F protein has been tested clinically (Gottlieb, J., et al. J Heart Lung Transplant (2016) 35:213-221). The longer half-life of a full IgG is advantageous compared to this precedent.

US/EU Pharm Chapter 1601 for Nebulizer Product Using Tidal Breathing provides regulatory guidance to qualify the delivered dose (USP Pharmacopeial Forum 36(2):534). A marketed nebulizer, the AeroNeb-Solo® device from Aerogen, Ltd (Deerfield, IL) uses a highly efficient vibrating mesh to generate the aerosol. Piezoelectric energy applied to the vibrational element at 150 MHz causes vibration of each of 1000 microscopic funnel shaped apertures within the mesh to act as a micropump drawing liquid through the holes producing a low velocity aerosol optimized for targeted drug delivery to the lungs. The low velocity minimizes shear forces that can denature proteins, making this system particularly useful for delivery of antibody therapeutics. The device typically delivers 9 times more aerosol dose than a standard small volume nebuliser such as a pressurized metered dose inhaler (as is typical for asthma treatment). Aerosol particle size is measured using a cascade impactor (In-Tox Products, Moriarity, NM). Using this system, we have produced aerosols of an antibody with a 2.17 μm mass median aerodynamic diameter (MMAD) and a 2.24 μm geometric standard deviation (GSD).

A second generation Aerogen product is specifically designed to treat patients requiring a ventilator. This Photo Defined Aperture Plate (PDAP) device provides significant improvements in ease of use as well as further dose reduction over the first generation product by achieving tighter control over the mean particle size. This is important because aerosol droplets that are too large do not penetrate deeply into the lungs, but if the droplets are too small, they don't settle out and are simply exhaled. The PDAP device also allows the nebulizer to be synchronized with the patient's breathing. Only generating the aerosol during the inspiration phase of the breathing cycle substantially improves the fraction of drug that is deposited in the lungs.

### Applications

The invention is also directed to pharmaceutical and veterinary compositions which comprise as active ingredients the binding moieties, mutants or other peptides or peptidomimetics of the invention. The compositions contain suitable physiologically compatible excipients such as buffers and other simple excipients. The compositions may include additional active ingredients as well, in particular in the case of immunogens immune system stimulants as vaccine adjuvants. The pharmaceutical or veterinary compositions may also contain other formulation excipients, including formulations for intra-nasal or inhaled delivery of mAbs as described in U.S. Pat. No. 9,718,875.

The immunogens are employed in a method to generate an immune response to RSV, comprising administering formulations containing them to a subject, including a human subject, such as a pregnant woman, an infant, an elderly human, or an immunocompromised subject. Infections related to RSV in other animal species may also be treated prophylactically by the immunogens or binding moieties of the invention.

### Example 1: Production of Proteins

### A. Production of Fab 3D3 and ScFv 2D10

Recombinant mAbs 3D3 and 2D10 were produced by transient-transfection in CHO cells and purification by immobilized protein A.

The Fab fragment of 3D3 was generated from recombinantly produced 3D3 by incubation with immobilized papain, followed by removal of the Fc fragment with immobilized protein A. Fab 3D3 was then purified by Superdex™ 200 size-exclusion chromatography media in 10 mM Tris-HCl pH 8.0 and 150 mM NaCl.

For recombinant scFv 2D10, a synthetic gene codon-optimized for Drosophila melanogaster encoding 2D10 heavy chain variable region, a (GGGGS) 3GGG linker (SEQ ID NO: 29), and 2D10 light chain variable region, was cloned into pMT-puro in-frame with an N-terminal BiP signal sequence and a C-terminal thrombin cleavage site followed by a Twin-Strep™ purification tag. The resulting scFv 2D10 expression plasmid was used to obtain stably-transfected Schneider 2 (S2) insect cells. Secreted scFv 2D10 was affinity purified on a StrepTrap™ column (biotin binding chromatography media), digested with thrombin protease to remove the purification tag, and then purified by Superdex™ 200 size-exclusion chromatography in 10 mM Tris-HCl pH 8.0 and 150 mM NaCl.

### B. Production of Epitopes

A synthetic gene encoding RSV G ectodomain (G[ecto]) P03423) was cloned into pCF in-frame with an N-terminal TPA signal sequence and C-terminal tandem 6-histidine and Twin-Strep™ purification tags. G[ecto] was produced by transient-transfection in CHO cells and secreted G[ecto] was affinity purified on a StrepTrap™ column.

A synthetic gene codon-optimized for Escherichia coli encoding RSV G residues 161 to 197 (G [161-197] ) with a C-terminal 6-histidine purification tag was cloned into pET52b. The peptide was expressed overnight in E. coli BL21 (DE3) at 18° C. The cells were then were lysed by ultrasonication in 20 mM Tris-HCl pH 8.0, 150 mM NaCl, and 25 mM imidazole (Buffer A) containing 2 μM MgCl₂, benzonase, and protease inhibitors. RSV G[161-197] was purified from soluble lysates by HisTrap™ FF affinity chromatography and eluted with a gradient into Buffer B (Buffer A containing 500 mM imidazole). Analogous methods were used to produce related peptides (G [162-172] and G [169-198] ).

### Example 2: ELISA Assays

Purified mAbs at a concentration of 5 μg/mL (150 μL total) are incubated overnight at room temperature in 96-well ELISA microtiter plates. Plates are then washed three times with PBS containing 0.05% Tween 20 (PBST). Wells are blocked by adding 150 μL of 5% BSA in PBS and incubating at room temperature for 1 hr followed by three PBST washes. Recombinant RSV G[ecto] at 5 μg/mL or RSV G [161-197] at 20 μg/mL in 1% BSA in PBS is serially diluted 1:3 with 1% BSA in PBS. Wells are incubated with 150 μL RSV G protein for 1 hr at room temperature, plates washed three times with PBST, and then incubated for 1 hr at room temperature with 150 μL HRP-conjugated-HisProbe (ThermoFisher Scientific) diluted 1:5000 in 1% BSA in PBS. Plates are washed three times with PBST and developed by adding peroxidase substrate o-phenylenediamine dihydrochloride (OPD) in 0.05 M phosphate-citrate buffer pH 5.0 and 1.5% hydrogen peroxide for 10 min at room temperature. The reactions are stopped by incubation with 2N sulfuric acid for 10 min at room temperature, and the absorbance measured at 490 nm. ELISA experiments are performed in biological triplicates.

### Example 3: Tests for Efficacy of Immunogens

Efficacy can be evaluated by injecting mice with a vaccine comprising RSV G or fragments thereof, an anti-G mAb that blocks the interaction of the G protein with CX3CR1, along with standard vaccine adjuvants; alternatively, administration may be by intra-nasal route (Kruijsen, D., et al. J Virol (2013) 87(13):7550-57). After adequate time for immunization, the mice are challenged with a virulent strain of RSV and observed for morbidity and mortality. The composition of the present invention results in protection of the subject mice. The immunogens of the invention are evaluated in a murine model using the following criteria.

TABLE 1
Immunogen Efficacy Criteria

| Category | Score | Detailed Criteria |
|---|---|---|
| RSV titers in lungs: | 4 | no detectable RSV |
| Based on pfu/g lung tissue at day 5 | 3 | 75% reduction of RSV in |
| post-challenge and RT-PCR | | lungs vs. naïve mice |
| quantitation of viral genomes/g | 2 | 50% reduction of RSV in |
| tissue | | lungs vs. naïve mice |
| | 1 | 25% reduction of RSV in |
| | | lungs vs. naïve mice |
| Lung Pathology: | 4 | No pathology |
| Based on the mean score for each | 3 | slight |
| parameter, i.e. peribronchiolar, | 2 | moderate |
| perivascular, interstitial and alveolar | 1 | severe |
| that involve each lung section | | |
| evaluated | | |
| Duration of immunity: | 3 | >6 months |
| Based on full protection from | 2 | 1-6 months |
| subsequent virus challenge | 1 | <1 month |
| Weight loss: | 3 | No weight loss |
| Parameter of morbidity | 2 | 5-19% weight loss |
| | 1 | ≥20% weight loss |
| Cellular immunity: | 3 | robust; Th1/Th2 balanced |
| Th1/Th2 assayed by IFNγ and IL-4 | 2 | moderate; Th1/Th2 balanced |
| ELISPOTs or by intracellular | 1 | Unbalanced Th2/Th1 |

cytokine FACS and by ELISA of
bronchial alveolar lavage fluid from
the lungs

### Example 4: Chemotaxis Assay

An in vitro assay for RSV G modulation of CX3CR1 measures receptor-mediated chemotaxis of human monocyte THP-1 cells (Tripp, R.A. et al. Nature Immunology (2001) 2:732-738). In this assay, recombinant RSV G[161-197] induced chemotaxis at levels equivalent to the entire RSV G ectodomain, an activity blocked by pre-incubation with 3D3 or 2D10 at a level comparable to that provided by anti-CX3CR1 polyclonal serum. Table 2 provides the results of this analysis, showing the inhibition of the biological activity by high affinity mAbs binding to the G CCD peptides.

TABLE 2
Chemotaxis Assay Results

| Negative | | | | | | RSV |
|---|---|---|---|---|---|---|
| Control | | | | RSV | RSV | G |
| (serum | Positive | RSV | RSV | G | G | [161-197] + |
| free | Control | G | G | [161-197] + | [161-197] + | anti- |
| media) | (+10% FBS) | [ecto] | [161-197] | 3D3 | 2D10 | CX3CR1 |
| 1.0 | 5.2 | 3.9 | 4.2 | 1.5 | 1.0 | 1.1 |

In more detail, the assay was performed using a transwell insert plate with an 8 um pore size. Approximately 2 million log-phase THP-1 cells (a human leukemia monocytic cell line) washed twice and suspended in serum-free RPMI 1640 media were added to the upper chamber of the insert plate. Negative control was serum-free media alone to which serum-free media containing 25 nM mAb was added to the lower chamber. As a positive control, media containing 10% FBS was added to the lower chamber. RSV G[ecto] or RSV G[161-197] samples were added to the lower chamber at a final concentration of 5 nM in serum-free media. For samples with RSV G[161-197] and mAbs, RSV G[161-197] was pre-incubated with 5-fold excess mAb (on a molar basis) for 20 min at room temperature, and then added to serum-free media in the lower chamber, for a final concentration of 5 nM RSV G[161-197] and 25 nM mAb. For samples with anti-CX3CR1 antibody, 2 μL 1 mg/mL anti-CX3CR1 rabbit polyclonal antibody (ThermoFisher Scientific Cat #PA5-19910) was incubated with THP-1 cells for 30 minutes in the upper chamber before being placed into the well. The assembled plates were incubated in a CO2 incubator at 37° C. for 5 h. Cells migrated to the lower chamber were counted, and the chemotactic indices were determined by comparing the fold-increase in cell migration toward the chemoattractant to cell migration toward serum-free media alone. Experiments were performed in at least four biological replicates.

What is claimed is:

1. An immunogen against respiratory syncytial virus (RSV) comprising a complex formed by an RSV G protein or peptidomimetic, wherein said RSV G protein includes a central conserved domain (CCD,) with a monoclonal antibody or antigen binding fragment thereof that blocks interaction of the recombinant RSV G protein with host receptor CX3CR1.

2. The immunogen of claim 1, wherein the CCD comprises residues 131-230 or 167-176 of said RSV G protein.

3. The immunogen of claim 1, wherein the monoclonal antibody or fragment has an affinity associated with a Kd of less than about 1 nM.

4. The immunogen of claim 1, wherein the monoclonal antibody comprises:
SEQ ID NO: 2 and SEQ ID NO: 16; or
SEQ ID NO: 4 and SEQ ID NO: 18; or
SEQ ID NO: 6 and SEQ ID NO: 20; or
SEQ ID NO: 8 and SEQ ID NO: 22.

5. A vaccine comprising the immunogen of any of claims 1-4.

6. The vaccine of claim 5 for use in a method of prophylaxis of respiratory syncytial virus (RSV) infection.

7. A vaccine, which comprises an mAb directed to the RSV G protein, which mAb includes an Fc modified to provide an extended effective lifetime in a subject.

8. The vaccine of claim 7, wherein the Fc portion binds to FcRn or mucin.

9. The vaccine of claim 7, wherein the Fc portion comprises a YTE mutation.

10. The vaccine of any of claims 7-9 for use in a method of prophylaxis or treatment of respiratory syncytial virus (RSV) infection.

11. A vaccine for prophylaxis or treatment of RSV formulated for inhalation.

12. The vaccine of claim 11 which comprises the immunogen of any of claims 1-4 or the modified mAb described in any of claims 7-9.

13. A method for prophylaxis or treatment of RSV infection in a subject, which method comprises effecting expression, in the cells of said subject, of an mAb or antigen-binding fragment thereof directed against the RSV G protein.

14. The method of claim 13 wherein the mAb includes an Fc modified to provide an extended effective lifetime in a subject.

15. The method of claim 13, wherein the mAb comprises:
SEQ ID NO: 2 and SEQ ID NO: 16; or
SEQ ID NO: 4 and SEQ ID NO: 18; or
SEQ ID NO: 6 and SEQ ID NO: 20; or
SEQ ID NO: 8 and SEQ ID NO: 22.


SEQUENCE LISTING (29 sequences)

SEQ ID NO: 1 - protein, 127 aa, Homo sapiens
  CHAIN 1..127: note = heavy chain of respiratory syncytial virus antibody 1F12
QVQLVQSESEVKKPGASVTVSCKPSGYTFNTYYIHWVRQAPGQGLEWMGVINPSGGSTTYTQRFQDRVTMTTDTSTNTIYMDLTSLRSDDTAMYYCVRGSNLLPHLWEWKPSHFDSWGQGTLVTVSS

SEQ ID NO: 2 - protein, 126 aa, Homo sapiens
  CHAIN 1..126: note = heavy chain of respiratory syncytial virus antibody 3G12
QLQLQESGPGLVKPSETLSLTCTVSGGSISSSNYYWGWIRQPPGKGLEWIASIHDSGSIYYNPSLRSRVTISVDTSKNQFSLKLSSVTAADTAVYYCARHLVWFGELRNNWFDPWGQGTLVTVASA

SEQ ID NO: 3 - protein, 124 aa, Homo sapiens
  CHAIN 1..124: note = heavy chain of respiratory syncytial virus antibody 1A5
QLQLQESGPGLVKPSETLSLTCTVSGDSITSGQYYWAWIRQAPGKGLEWIGSIHYSGSTYQNPSLKSRLTISVDTSRDQISMKLSSVTVAESAVYYCARQQLSLSPVENWFDPWGQGTLVTVSS

SEQ ID NO: 4 - protein, 124 aa, Homo sapiens
  CHAIN 1..124: note = heavy chain of respiratory syncytial virus antibody 3D3
EEQLVESGGGLVQPGRSLRLSCVGSGLRFEEHAMHWVRQAPGRGLEWVSGISWNSGSVGYADSVKGRFTTSRDNAKDILFLEMNTLRSEDTALYFCAIMVATTKNDFHYYKDVWGKGTTVTVSS

SEQ ID NO: 5 - protein, 124 aa, Homo sapiens
  CHAIN 1..124: note = heavy chain of respiratory syncytial virus antibody 1G1
QVHLVQSGVEVKKPGASVRVSCKASGYTFATYGITWVRQAPGRGLEWVGWITPYNDRTSYAQIFHGRVTMTTDTSTNTAYMELRSLRSDDTAMYYCARNHCNFYHDFWSGLDYWGQGTLVSVSS

SEQ ID NO: 6 - protein, 121 aa, Homo sapiens
  CHAIN 1..121: note = heavy chain of respiratory syncytial virus antibody 2B11
QVQLVQSGAEVKKPGSSVKVPCKASGGTFSTYPISWVRQAPGQGLEWMGRIIPDPPMANIAQKFQGRVSFSADKSTTIVYMELSSLRSEDTAVYFCAREILQSPPFAVDVWGQGTMVAVSS

SEQ ID NO: 7 - protein, 127 aa, Homo sapiens
  CHAIN 1..127: note = heavy chain of respiratory syncytial virus antibody 5D8
QAQLVESGGGVVQPGRSLRLSCAASGFTFSYSGMHWVRQAPGRGLEWLAVISYDGNKMYYADSVKGRFTISRDNSKMTLYLQMNSLRREDTAVYYCAKDGLDYGGDLVYYGMDVWGNGTTVTVSSAS

SEQ ID NO: 8 - protein, 126 aa, Homo sapiens
  CHAIN 1..126: note = heavy chain of respiratory syncytial virus antibody 2D10
QVQLVQSGPEVKKPGASVRLSCKASGYVFTNYGVSWVRQAPGQGLEWMGWSSPYNGNTYYAQKLKARVTMTTDTSTNTAYMELRSLRSDDTAVYYCGRDMLGVVQAVAGPFDSWGQGTLVTVSSAS

SEQ ID NO: 9 - protein, 126 aa, Homo sapiens
  CHAIN 1..126: note = heavy chain of respiratory syncytial virus antibody 3F9
QVQLVQSGGEVKRPGASVRVSCKASGYTFINYAISWVRQAPGQGLEWLGWISAYNGNTHYAQKVQDRVTMTTDASTTTAYMELRGLKSDDTAVYSCARLPLLGYSSGWYAFDMWRQGTMVPVSSAS

SEQ ID NO: 10 - protein, 127 aa, Homo sapiens
  CHAIN 1..127: note = heavy chain of respiratory syncytial virus antibody 1D4
QVQLLQSGAELKKPGASVKVSCQASIDTFSTYYIHWVRQAPGQGLEWLGVINPSGGSTTYAQKFQDRITLTTDTSTRTVYMDLSSLRSEDTAVYYCARVHKGRAEQWQLLHGHFDLWGRGSLVTVSS

SEQ ID NO: 11 - protein, 126 aa, Homo sapiens
  CHAIN 1..126: note = heavy chain of respiratory syncytial virus antibody 1G8
QLQLQESGPGLVKPSETLSLTCTVSGDSITSGQYYWAWIRQAPGKGLEWIGSIHYSGSTYQNPSLKSRLTISVDTSRDQISMKLSSVTVAESAVYYCAKQQLSLSPVENWFDPWGQGTLVTVSSAS

SEQ ID NO: 12 - protein, 120 aa, Homo sapiens
  CHAIN 1..120: note = heavy chain of respiratory syncytial virus antibody 6A12
EVQVVESGGGLVQPGGSLRLSCVASGFTFSSYWMHWVRQAPGKGLVWVSRIYSDGSSTTYADSVKGRFTISRDNAKNTLYLQMNSLRAEDTAVYYCVRVLGAAMFDIWGQGTVVTVSSAS

SEQ ID NO: 13 - protein, 128 aa, Homo sapiens
  CHAIN 1..128: note = heavy chain of respiratory syncytial virus antibody 10C6
QVQLVESGGGVVQPGRSLRLSCEASGFTFSGYAMHWVRQAPAKGLEWVAVISFDGSNNYYADSVKGRFTISRDNSKNMLFLQMNSLRAEDTAVYYCVRPDVIAVAGTALSNPFDLWGLGTMVTVSSAS

SEQ ID NO: 14 - protein, 122 aa, synthetic construct
  REGION 1..122: note = synthetic construct
  CHAIN 1..122: note = humanized heavy chain of respiratory syncytial virus antibody Hu 131-2G
QVQLVQSGAEVKKPGASVKVSCKASGYSFTGFTMNWVRQATGQGLEWMGLINPFNGNTGYNQKFKGRVTMTRNTSISTAYMELSSLRSEDTAVYYCARSGKSYDYEAWFTYWGQGTLVTVSA

SEQ ID NO: 15 - protein, 109 aa, Homo sapiens
  CHAIN 1..109: note = light chain of respiratory syncytial virus antibody 1E12
EIVLTQSPGTLSLSPGERATLSCRASQSITNNYLAWYQQKPGQAPRLLIYDSFSRATGIPERFSGSGSGTDFTLTISRLEPEDFAVYYCQHYVRSPLTFGPGTKVEIKR

SEQ ID NO: 16 - protein, 107 aa, Homo sapiens
  CHAIN 1..107: note = light chain of respiratory syncytial virus antibody 3G12
EIVMTQSPATLSVSPGERATLSCRASQSVNSNLAWYQHKPGQAPRLLIYGASTRATGIPARFSGSGSGTDFTLTISSLQSEDFAVYYCQQYNNWPLFGPGTKVDLKR

SEQ ID NO: 17 - protein, 109 aa, Homo sapiens
  CHAIN 1..109: note = light chain of respiratory syncytial virus antibody 1A5
EIVVTQSPVTLSVSPGESATLSCRSARSVGSRLAWYQQKPGQPPRLLIFAASTRATGIPARFSGSGSGTDFTLIISGLQSEDYAVYYCQQYKEWPLFTFGPGTTVDSKR

SEQ ID NO: 18 - protein, 106 aa, Homo sapiens
  CHAIN 1..106: note = light chain of respiratory syncytial virus antibody 3D3
QIVLTQSPATLSLSPGERATLSCKASQSVSNHLAWYQQKPGQAPRLLIYETSNRATGIPPRFSGSGSGTDFTLTISSLEPEDFAVYYCQQRNNWYTFGQGTKLEIK

SEQ ID NO: 19 - protein, 108 aa, Homo sapiens
  CHAIN 1..108: note = light chain of respiratory syncytial virus antibody 1G1
SFELTQPPSVSVSPGQTARITCSGDALPKQYVYWYQQKPGQAPVLVIYKTTERPSGIPERFSDSSSGTTVTLTISAAQAEDEADYYCQSVDSSGTYVFGIGTKVTVLG

SEQ ID NO: 20 - protein, 113 aa, Homo sapiens
  CHAIN 1..113: note = light chain of respiratory syncytial virus antibody 2B11
QSALTQPASVSGSPGQSITISCTGSSSDVGGYSHVSWYQQHPGKVPKLIISEVSNRPSGISNRFSGSKSANTASLTISGLQPEDEADYYCGSYASTNILHYVFGTGTKVTVLS

SEQ ID NO: 21 - protein, 114 aa, Homo sapiens
  CHAIN 1..114: note = light chain of respiratory syncytial virus antibody 5D8
SYELTQPPSVSVSPGQTGRITCTGSEASGDALASRYAYWYQHKSGQAPVVLIYKDTERPSGISERFSGSSSGTTVTLIISGVLAEDEADYYCKTSVRNGTSWVFGTGTMLTVLR

SEQ ID NO: 22 - protein, 108 aa, Homo sapiens
  CHAIN 1..108: note = light chain of respiratory syncytial virus antibody 2D10
DTPMTQSPSSVSASVGDRVTISCRASQGISNSLAWYQQKLGKAPQLLIYAASSLQSGVPSRFSGSGSGTDFTLTISSLQPEDFATYYCQQTNTFPFTFGPGTKVEVRR

SEQ ID NO: 23 - protein, 111 aa, Homo sapiens
  CHAIN 1..111: note = light chain of respiratory syncytial virus antibody 3F9
QSALTQPPSASGSPGQSVTISCTGTSSDVGGYNYVSWYQQHPGKAPKLMIYEVNKRPSGVPDRFSGSKSGNTASLTVSGLQAEDEAEYYCSSYAGSMNWVFGGGTKLTVLG

SEQ ID NO: 24 - protein, 114 aa, Homo sapiens
  CHAIN 1..114: note = light chain of respiratory syncytial virus antibody 1D4
DIVMTQSPDSLAVSLGERATINCKSSQSVLYSSNNKTYLAWYQQKPRQPPELLIYWASTRESGVPDRFSGSGSGTNFTLTISSLQAEDVAVYYCQQYYTTPYTFGQGTKLEIKR

SEQ ID NO: 25 - protein, 109 aa, Homo sapiens
  CHAIN 1..109: note = light chain of respiratory syncytial virus antibody 1G8
EIVVTQSPVTLSVSPGESAALSCRASRSVGSRLAWYQQKPGQPPRLLIFAASTRATGIPARFSGSGSGTDFTLIISGLQSEDYAVYYCQQYKEWPLFTFGPGTTVDSKR

SEQ ID NO: 26 - protein, 110 aa, Homo sapiens
  CHAIN 1..110: note = light chain of respiratory syncytial virus antibody 6A12
EIVLTQSPATLSLSPGERATLSCRASQSVSSYLAWYQQKPGQAPRLLISDASNRATGIPPRFSGSGSGTDFTLTISSLEPEDFAVYYCQQRSNWPPRFTFGPGTIVDIRR

SEQ ID NO: 27 - protein, 107 aa, Homo sapiens
  CHAIN 1..107: note = light chain of respiratory syncytial virus antibody 10C6
EIVMTQSPATLSVSPGERALSCRASQSVRSNLVWYQQKPGQAPRLLIFGASTRATGIPARFSGSGSGTEFTLTISSLQSEDFALYFCQQNNNWPPTFGQGTRLEIKR

SEQ ID NO: 28 - protein, 113 aa, synthetic construct
  REGION 1..113: note = synthetic construct
  CHAIN 1..113: note = humanized light chain of respiratory syncytial virus antibody Hu 131-2G
DIVMTQTPLSLSVTPGQPASISCRSSQTIVHTNGNTYLEWYLQKPGQSPQLLIYKVSNRFSGVPDRFSGSGSGTDFTLKISRVEAEDVGVYYCFQGSHVPFTFGSGTKLEIKR

SEQ ID NO: 29 - protein, 18 aa, synthetic construct
  REGION 1..18: note = synthetic construct
  REGION 1..18: note = (GGGGS)3GGG linker
GGGGSGGGGSGGGGSGGG